(19) United States
(12) Patent Application Publication
(10) Pub. No.: US 2023/0191037 A1
(43) Pub. Date: Jun. 22, 2023

(54) DRUG DELIVERY DEVICE WITH CLICK SOUND DURING DELIVERY

(71) Applicant: Novo Nordisk A/S, Bagsvaerd (DK)

(72) Inventor: Kurt Solgaard, Graested (DK)

(73) Assignee: Novo Nordisk A/S, Bagsvaerd (DK)

(21) Appl. No.: 17/925,546

(22) PCT Filed: May 12, 2021
(86) PCT No.: PCT/EP2021/062590

§ 371 Date: Nov. 15, 2022

(30) Foreign Application Priority Data
May 18, 2020 (EP) 20175119.5

(51) Int. Cl. A61M 5/315

(52) U.S. Cl. CPC A61M 5/31505; A61M 5/31585; A61M 2205/581

(57) ABSTRACT

A drug delivery device (100) for delivering an amount of medicament comprising a housing and a medicament reservoir (135) with a piston (136). The device further comprises a ratchet mechanism comprising a first and a second set of movable and stationary ratchet members (281, 381, 481, 165.1, 365.1) adapted to provide a first and a second plurality of periodic audible signals, during the expelling of medicament. The ratchet mechanism is further adapted for generating the first and second plurality of periodic signals out-of-phase, whereby each of the signals are distinguishable by a user. The ratchet mechanism further comprises means to provide a radial force to counteract influence from radial play, which otherwise may generate variation in the frequency.

The present invention relates to a drug delivery device and a method of using the drug delivery device for delivering an amount of medicament. The invention further relates to such a drug delivery device comprising a ratchet mechanism for signaling the delivery of medicament and a method of using the device.

### BACKGROUND OF THE INVENTION

Drug delivery devices for self-administration of different liquid drug formulations presently exist in various shapes and sizes. Some are adapted for connecting to an infusion set, and some are connectable or integrated with an injection needle. The latter type is referred to as injection devices. Some are durable devices comprising a cartridge with a drug reservoir, wherein the cartridge can be changed. Others are disposable devices that are discarded when the cartridge is empty. Disposable devices can be either multi-dose or single dose devices, in which the user can set the desired dose size prior to each injection, or the user can activate the delivery a preset fixed dose.

The drive mechanism of a drug delivery device typically comprises a rotatably arranged drive member coupled to a piston rod. When the element rotates, the piston rod is advanced to deliver an amount of drug from a reservoir. The drive member can be axially splined to the piston rod, wherein the piston rod is threadably engaged with the housing. See for example WO 2020089167 and WO 14161952 filed by Novo Nordisk. Alternatively, the drive member can be threadably enagaged with the piston rod, and axially splined to the housing. Both alternatives provide an axial movement of the piston rod in response to rotation of the drive member. The drive member can be rotated by a spring, an electric motor, or a plunger arranged to be manually driven by a user. See for example wo18007259 file by Copernicus.

As an alternative to a rotatable drive member, the piston rod can be in threaded engagement with the drive member and the housing, and a forced axial non-rotatable movement of the drive member can induce a rotation and axial movement of the piston rod, i.e., a helical movement, see for example the embodiment of FIGS. 1-16 of WO04078239 filed by DCA.

As a further alternative the piston rod can be rotationally locked to the housing. During dose setting the, the drive member is helically moved along an outer thread of the piston rod. During dispensing both the drive member and the piston rod are moved axially without rotation, see for example WO05018721 filed by Eli Lilly.

Devices adapted for delivering a given fixed dose are preferred by some users, since they may not feel comfortable with or be capable of operating the device to adjust the correct dose each time. When devices for instance are used by children or older people, simplicity and ease of use is important to avoid user error leading to over- or under dosing. In other cases, the treatment regimen prescribes a fixed dose of e.g. a GLP-1 type of drug. Other users prefer an injection device allowing the dose to be adjusted.

In order to provide acoustic feedback to the user the drive mechanism is equipped with one or more click arms that rotate with the rotating drive member. The free end of the arm is spring loaded against stationary saw tooth shaped ratchet teeth arranged in a circular pattern on a stationary part. Alternatively, the rotating drive member is provided with ratchet teeth and a stationary housing part with one or more deflectable click arms.

WO 2020089167 discloses an injection device, wherein the user can only perform an injection once a minimum or fixed dose has been set. The document describes that the torque of a torsion spring can transfer a rotation of the piston rod driver 65. The piston rod driver is further provided with one or more one-way arms 67 engaging a toothed periphery 59 inside the base part 55 of the housing structure only allowing rotation of the piston rod driver 65 in one rotational direction (anti clock-wise in the disclosed example when viewed form a proximal position). During rotation in the counter clock-wise direction the one-way arms 67 clicks over the toothed periphery 59 of the base part 55 of the housing structure. This provides a distinct sound to the user that a dose is being distributed.

U.S. Pat. No. 7,758,550 filed by Techpharma discloses an injection device for administering a liquid product in a single fixed dose. The device comprises a drive mechanism with a piston rod 5 for automatically injecting the liquid product. The device further comprises a catch sleeve 22 and a catch 30 comprising a number of latching elements 31 (FIG. 11) arranged along an axial or longitudinal axis of the device. The catch sleeve is relatively moveable to the catch during axial movement of a piston rod 5, and thereby during expelling of the dose. The relative axial movement between the engaging member and the catch generates a haptic and/or acoustic signal during expelling of the dose.

WO 14161952 describes an injection device allowing the dose to be adjusted for each injection. The drive mechanism for dispensing the dose is spring driven and based on the same principle as described in WO2020089167, and WO14161952 describes that the drive member comprises a pair of opposed circumferentially extending flexible ratchet arms adapted to engage a ring-formed 10 array of one-way ratchet teeth 205. During dose delivery, the drive member rotates anticlockwise and the ratchet arms 235 also provide the user with small clicks due to the engagement with the ratchet teeth 205, e.g. one click per unit of insulin expelled. In the shown embodiment 24 ratchet teeth are provided corresponding to 15 degrees rotation per unit of insulin. The ratchet arms 235 provide the user with small clicks due to the engagement with the ratchet teeth 205, e.g. one click per unit of 15 insulin expelled.

WO 99/38554 filed by Novo Nordisk discloses an injection device with a dose setting mechanism and a manually driven drive mechanism. FIG. 1-5 illustrates a first embodiment of such a device comprising a housing 1 having an internal thread 5 for mating an externa thread of a piston rod 6, and a piston rod guide 14 splined to the piston and adapted for driving the piston rod 6. The inner surface of the housing is further provided with pawl wheel teeth 10. At least one pawl 13 mounted on the piston rod guide 14 cooperates with the pawl teeth 10 so that said piston rod guide can only be rotated clockwise. A dose is dispensed, in response to a clockwise rotation of the piston rod guide. FIG. 6-10 illustrates a second embodiment of such a device. The device comprises an injection button 23 with an extension 33 adapted for driving the piston rid. A longitudinal bore 35 in the injection button and its extension 33 is provided with an internal helical rib 36 engaging a corresponding helical groove in an enlargement 37 at the proximal end of the piston rod to form a thread connection between the button 23 and the piston rod 6. The piston rod is also threadably engaged with the housing. A piston rod guide with a pawl 13 (FIG. 8) is splined to the piston rod, and axially locked to the housing. During dispensing, the injection button is rotationally locked, and induces a helical movement of the piston rod, in response to a distal axial movement. Hereby, the piston rod guide with the pawl 13 rotates and ensures uni-directional rotation. In such an embodiment the pawl is not attached to the drive member rotating the piston rod. On the contrary, the pawl is connected to the piston rod guide which is rotated by the piston rod, which is driven by the drive member.

U.S. Pat. No. 10,420,896 B2 owned by Sanofi, describes an injection device with a drive mechanism comprising a ratchet adapted to rotate in a dose decrementing direction during a dose dispensing procedure. The ratchet member comprises a radially extending arm which consecutively meshes with a toothed profile of the housing. The mutual engagement of the ratchet member 86 sliding along the toothed profile also generates an audible click sound inherently indicating to tile user, that the dosing procedure is in progress.

The document further describes a second ratchet mechanism for incrementally adjusting a dose. The second ratchet mechanism is therefore decoupled, when the injection device is in the dispensing mode. The second ratchet mechanism comprises first and second ratchet elements which are circumferentially off-set by half of a period of consecutively arranged teeth. In this way, the size of discrete steps for setting of a dose can be effectively reduced without the necessity to make use of respective small sized teeth and ratchet elements.

For some applications, when the rotational speed of the piston rod or the drive member is relatively low, the frequency of the signaling clicks indicating that a dose is being expelled is too low. This can for example be for applications with a high pitch on the piston rod, providing a relatively large axial displacement relative to the angular rotation.

To some extent the clicking speed can be increased by fitting a larger number of smaller/shorter teeth. Because there are practical limits to how small teeth can be made the achievable increase of click speed is limited in the traditional and typical setting described above.

For other applications, for example for injection devices expelling high viscosity liquids and or injecting through injection needles with a small diameter, the frequency may also be too low.

WO2019110618 filed by Novo Nordisk discloses a drug injection device comprising a first element 140 which rotates during expelling and a second element which is stationary. The stationary element 103 comprises a first and a second deflectable arm with a first and second tip, respectively. The tip portion 103a′ is located generally diametrically opposite from the tip portion 103b′. However, in accordance with the invention, the tip portions 103a′ and 103b′ are located on the second element 103 so that the tip portions 103a′ and 103b′, by cooperating with diametrically opposed protrusions 143 of the first element 140, will not assume the biased radial second position at the same time, but slightly offset from each other. In the shown embodiment, the tip portions 103a′ and 103b′ are located approximately 178 degrees apart so that, as the first element 140 rotates relative to the second element 20 103, the first deflectable arm 103a will experience cooperation with a particular first protrusion slightly before the second deflectable arm 103b will experience cooperation with a protrusion arranged diametrically opposite from the first protrusion. As the deflectable arms are diametrically arranged corresponding diametrically arranged piezoelectric elements can measure the deflection. As the tips are slightly offset there will be a time delay between registered deflections of the first and second arm. A processor is connected with the piezoelectric elements to register generated activation signals and can determine the amount of drug. A time delay between pulses from the first and second deflectable arm can be used to detect correct functioning.

WO 03/008023 filed by Eli Lilly discloses a medication dispensing apparatus comprising a priming driver 100 with a grip portion 102. A driver body portion 104 extends from the grip portion 102. The driver body portion is sized to be inserted within the interior hollow of a housing body 62, and is further adapted to threadably engage a drive screw 80 through thread segments 132. The drive screw 80 is rotationally locked to the housing through longitudinal slots 90. Therefore, rotation of the driver 100 advances the drive screw. Within a proximal region of the body portion 104, at least one pawl is formed which cooperates with the ratchet teeth 68 on the housing 62 to limit the rotation of a driver 100 relative to housing 60 to a single direction. In the shown driver 100, a pair of nearly diametrically opposed pawls are provided in the form of angularly extending, radially bendable pawl fingers 106 having catch ends 108 that extend sufficiently far radially outward to engage ratchet teeth 68. By slightly offsetting the pawls so as to not be precisely, diametrically opposed, as shown, one catch end 108 can engage a ratchet tooth 68 while the other catch end 108 is being ramped inward by contact with the middle of a different ratchet tooth 68, whereby the angular precision of the offset pawls is twice as good as if lined up diametrically. Medication is expelled when the driver 100 is rotated in the allowed direction during priming.

The present invention concerns solutions to how the clicking speed can be increased during delivery of a dose. It is therefore an object of the present invention to provide drug delivery devices with a signalling mechanism providing a desirable number and consistency of signals during expelling in a simple and cost-effective manner, and without compromising design and production limitations. It is a further object of the invention to provide a dose clicking mechanism preventing an unpredictable click pattern causing the click pattern to vary in an unpredictable manner.

### DISCLOSURE OF THE INVENTION

In the disclosure of the present invention, embodiments and aspects will be described which will address one or more of the above objects or which will address objects apparent from the below disclosure as well as from the description of exemplary embodiments.

In a first aspect of the present disclosure is provided a drug delivery device for delivering an amount of medicament, wherein the device comprises:
a housing;
a medicament reservoir with a piston;
a drive mechanism comprising: a piston rod for driving the piston during a distal movement and thereby expelling the amount of medicament, a drive member operationally arranged for driving the piston rod, and a rotatably arranged movable ratchet body, wherein the movable ratchet body is operatively connected to the piston rod and adapted to rotate relative to the housing during delivery of the medicament, wherein the housing comprises a stationary ratchet body arranged to cooperate with the movable ratchet body and thereby provide a ratchet mechanism allowing a ratcheted guiding of the piston rod during the expelling of drug;

wherein the ratchet mechanism comprises a first and a second set of movable and stationary ratchet members adapted to provide a first and a second plurality of periodic audible signals, corresponding to the first and the second set of ratchet members, in response to a relative rotational movement between the movable and the stationary ratchet body, during the expelling of medicament;

wherein the ratchet mechanism is adapted to generate the first and second plurality of periodic signals out-of-phase, whereby each of the signals are distinguishable by a user, wherein a radial play is provided between the movable ratchet body and the housing, and wherein the movable ratchet body or the housing is adapted to provide a biasing radial reaction force between the movable ratchet body and the housing to counteract influence by the radial play, and thereby to reduce a variation of a frequency of the first periodic signal or the second periodic signal.

Hereby is provided a clicking mechanism comprising to sets of periodic click generating sets of ratchet members, wherein each set comprises a movable ratchet member and a stationary ratchet member. The movable members are provided on a movable ratchet body and the stationary members are provided on the housing which also provides the stationary ratchet body. The radial biasing force between the two ratchet bodies compresses the bodies in a radial direction and creates a reaction force, whereby influence of radial play is reduced, in the sense that time intervals with an otherwise increasing frequency for each of the sets of ratchet members is reduced or eliminated.

In a further aspect, tips of the ratchet members of the movable ratchet body are positioned within an angular distance of 45 degrees to provide a radial force against the housing, and thereby counteract the influence of the radial play.

In a further aspect, tips of the ratchet members of the movable ratchet body are positioned within an angular distance of 10 degrees to provide a radial force against the housing, and thereby counteract the influence of the radial play.

In a further aspect, the movable ratchet body comprises a spring element providing a radial force against the housing, and thereby counteracts the influence of the radial play.

In a further aspect, the housing comprises a spring element providing a radial force against the movable ratchet body, and thereby counteracts the influence of the radial play.

In a further aspect, the piston rod is in threaded engagement with the housing and the drive member is axially splined to the piston rod, wherein a rotational movement of the drive member induces an axial movement of the piston rod during the expelling of medicament.

In a further aspect, the piston rod s axially splined to the housing and the drive member is in threaded engagement with the piston rod, wherein a rotational movement of the drive member induces an axial movement of the piston rod during the expelling of medicament.

In a further aspect, the movable ratchet body is the drive member, wherein the audible signals are generated by rotation of the drive member together with the piston rod.

In a further aspect, tips of the ratchet members of the movable ratchet body are positioned within an angular distance between 170 and 190 degrees.

In a further aspect, tips of the movable ratchet members are axially aligned, whereby tip contact points on the stationary ratchet member are axially aligned.

In a further aspect, the piston rod is in threaded engagement with the housing and the drive member is in threaded engagement with the piston rod, wherein an axial movement of the drive member induces a rotational and axial movement of the piston rod during the expelling of medicament, wherein the movable ratchet body is provided as a portion of a piston rod guide axially splined to the piston rod, wherein the audible signals are generated by rotation of the piston rod together with the piston rod guide.

In a further aspect, the ratchet mechanism is adapted to allow one-directional rotation of the movable ratchet body, and thereby inhibit proximal movement of the piston rod.

In a further aspect, the drive mechanism is adapted to generate the periodic signals with a constant ratio between the frequency of the first periodic signal and the second periodic signal.

In a further aspect, the drive mechanism is adapted to generate the first and the second periodic signals with the same frequency.

In a further aspect, the drive mechanism is adapted to generate the periodic signals in anti-phase, to provide a consistent sound generation during expelling and a homogeneous resolution in time.

### BRIEF DESCRIPTION OF THE DRAWINGS

In the following embodiments of the invention will be described with reference to the drawings:

FIG. 1 illustrates an exploded view of a fixed dose drug delivery device with multiple doses according to a first embodiment of the present disclosure.

FIG. 2A illustrates a perspective view of an inner tubular portion of the housing and the drive tube of the device of FIG. 1.

FIG. 2B illustrates a cross section along the indicated line A-A and viewed from the proximal end.

FIG. 2C illustrates the cross section A-A viewed from the distal end.

FIG. 3A, illustrates in perspective view the drive tube with a flexible ratchet member of the device of FIG. 1.

FIG. 3B illustrates the spring base with the angularly directed track of saw-tooth shaped teeth of the device in FIG. 1.

FIG. 3C illustrates in perspective view the drive tube inserted into a tubular portion of the spring base of the device of FIG. 1.

FIG. 4 illustrates operation of the drug delivery device of FIG. 1.

FIG. 5A illustrates the working principle of an embodiment of a drive tube with movable ratchet members according to the present disclosure. The ratchet members are axially aligned. Box A illustrates a cross sectional view and box B illustrates an unfolded perspective view.

FIG. 5B illustrates the working principle of an alternative embodiment of a drive tube with movable ratchet members according to the present disclosure. The ratchet members are arranged with an axial off-set.

FIG. 5C illustrates the working principle of an alternative embodiment of a drive tube with movable ratchet members according to the present disclosure. The movable ratchet members are arranged with an axial off-set, and the housing comprises two stationary ratchet members.

FIG. 6 illustrates a perspective view of a drive tube of an embodiment, wherein the working principle for the embodiment is illustrated in FIG. 5A. The ratchet members are arranged with an angle of approximately 180 degrees and are axially aligned.

FIGS. 7A and 7B illustrate a further development of the embodiment of FIG. 6, wherein radial play has been limited or eliminated by adding an integrated spring. The working principle of the embodiment is illustrated in FIG. 5A. The ratchet members are axially aligned and a spring minimizes the effect from radial play.

FIG. 8 illustrates a perspective view of a drive tube of an embodiment, wherein the working principle for the embodiment is illustrated in FIG. 5B. The ratchet members are arranged in close angular proximity and with an axial off-set.

FIG. 9 illustrates a perspective view of a drive tube of an embodiment, wherein the working principle for the embodiment is illustrated in FIG. 5A. The ratchet members are arranged in close angular proximity and are axially aligned off-set.

In the figures like structures are mainly identified by like reference numerals. Reference numbers followed by the letter “a” is used to denote the distal end of the structure, and numbers followed by “b” is used to denote the proximal end. Reference numbers comprising a first number followed by a “.” and a second number is used to denote a functional or structural detail of a structure. In this way the first number indicates a primary (relatively large) structure and the second number indicates a secondary (relatively small) structure or a specific function. Reference numbers followed by the letters c, d and e indicate features with rotational symmetry or features rotationally shifted.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member is used for a given component it can be used to define a unitary component or a portion of a component, having one or more functions.

In the following detailed description, numerous specific details are set forth in order to provide a thorough understanding of the present disclosure. However, it will be apparent to one of ordinary skill in the art that the present disclosure may be practiced without these specific details.

It will also be understood that, although the terms first, second, etc. may be used herein to describe various elements or positions, these elements or positions should not be limited by these terms. These terms are only used to distinguish one element or position from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the term distal and proximal end is in analogy with the terminology from anatomy used to describe the end situated away from or nearest the point of attachment to the body. Therefore, the distal end of an injection device is defined in a context, where a user holds the device in a ready to inject position, whereby the end with the injection needle will be the distal end and the opposite end will be the proximal end. Furthermore, distal and proximal ends of individual components of the device is also defined in that context.

As used herein, rotational symmetry, is a property of a structure when it appears the same or possess the same functionality after some rotation by a partial turn. A structure's degree of rotational symmetry is the number of distinct orientations in which it appears the same for each rotation. Rotational symmetry of order n, wherein n is 2 or more, is also called n-fold rotational symmetry, or discrete rotational symmetry of the n^{th} order, with respect to a particular point (in 2D) or axis (in 3D), which means that rotation by an angle of 360°/n does not change the object. The property of the structure may both relate to the visible appearance and the functional capability of structural feature.

As used herein, the term clockwise direction is used to describe the direction that the hands of a clock rotate as viewed from in front. Therefore, the clockwise rotation of the injection device is the clockwise rotation observed, when viewing the device from the distal face, i.e., when viewed in the proximal direction. Counterclockwise or anticlockwise rotation is defined as the opposite direction.

As used herein, the proximal face is the face of the device as viewed from the proximal end and in the distal direction, wherein a distal face is the face of the device as viewed from the distal end and in the proximal direction.

As used herein, a positive axial or longitudinal direction is defined from the proximal end towards the distal end. A positive axial direction and a distal direction are used interchangeably with the same meaning. Similar, the definitions of a negative axial direction and a proximal direction are used interchangeably with the same meaning. A central axis of the device is defined through the centre of the injection device in the positive axial direction, which is also referred to as a longitudinal axis, with the same meaning.

As used herein, a positive radial direction is defined along a radial axis originating at the central axis and with a direction perpendicular to the central axis.

A positive circumferential or positive angular direction is defined for a point positioned at a radial distance from the central axis, wherein the positive circumferential direction is the counterclockwise direction when observed in the negative axial direction. The circumferential direction is perpendicular to the axial and radial direction.

Both the radial and the circumferential direction are herein referred to as transverse directions, as they are transverse or normal to the axial direction. The transverse plane is herein defined as a plane spanned by two vectors in the radial and circumferential direction, and with the central axis as the normal vector.

As used herein, axial movement of a structure is used to describe a movement, wherein the displacement vector of the structure has a component in the axial direction. A translational movement is used to describe a uniform motion in the axial direction only. A pure, strict or uniform axial movement is the same as a translational movement and the terms are used interchangeably.

Radial movement of a structure is used to describe a movement, wherein the displacement vector of the structure has a component in the radial direction. A pure or strict radial movement is used to describe a uniform motion in the radial direction only. Thus a pure, strict and uniform radial movement is the same and the terms are used interchangeably.

Circumferential or rotational movement of a structure is used to describe a movement, wherein the displacement vector of the structure has a component in the circumferential direction. A pure or strict circumferential movement is used to describe a uniform motion in the circumferential direction only. Thus a pure, strict and uniform circumferential movement is the same as pure, strict and uniform rotational movement, and these terms are used interchangeably. The definition of rotational movement for a structure also encompasses the special case, wherein the structure comprises a central axis defining the axis of rotation. In this special case, all the positions of the structure, which are off the central axis, are subject to a circular circumferential movement, whereas the displacement vector of the positions on the central axis is zero. Therefore, a structure rotating about its own central axis is said to perform a rotational movement.

A helical movement of a structure is used to describe a combined axial and rotational movement, wherein the displacement vector of the structure comprises a circumferential and an axial component. The definition of helical movement for a structure also encompasses the special case, wherein the structure comprises a central axis defining an axis of rotation. In this special case, all the positions of the structure, which are off the central axis, are subject to a helical movement, whereas the displacement vector of the positions on the central axis only comprises an axial component. Therefore, a structure rotating about its own central axis and moving in an axial direction is said to perform a helical movement.

In this context pure, strict and uniform movements are abstract mathematical definitions, and these terms are used to describe an ideal or abstract movement of the structures. Therefore, a structure in a real device should not be expected to exhibit this ideal behaviour, rather such a structure should be expected to move in a pattern approximating such an ideal movement.

As used herein a right-handed thread or helical portion is a thread or helix portion whose helix moves in the positive axial direction, when a screw with the thread is turned counterclockwise.

A screw with a right handed-thread is by convention the default thread, and is screwed in the positive direction by counterclockwise rotation usually performed by the right hand. Similar, a screw with a left-handed thread is screwed in the positive direction by a clockwise rotation, and can thus be performed with the left hand and mirror the movement of the right hand operating a right-handed thread.

The present disclosure describes a drug delivery device for delivering an amount of medicament, wherein the device comprises a housing, a medicament reservoir with a piston and a drive mechanism.

The drive mechanism comprises a piston rod for driving the piston and thereby it is adapted for expelling the amount of medicament through an outlet of the reservoir. The drive mechanism further comprises a drive member for driving the piston rod. The drive member is movably arranged in the housing and can be arranged to drive the piston rod in response to either of rotational or an axial movement. The drive mechanism further comprises a movable ratchet body, which is also movably arranged in the housing. The ratchet body can be provided as an integral or attached portion of the drive tube, or it can be a separate structure driven or moved by the piston rod, in response to expelling of medicament. In either way, the movable ratchet body is operatively connected to the piston rod and is adapted to move relative to the housing, when the piston rod moves relative to the housing during the expelling.

The housing comprises a stationary ratchet body cooperating with the movable ratchet body and thereby provides a ratchet mechanism adapted to allow a distal ratcheted movement of the piston rod, which is required in order to expel medicament from the reservoir. Furthermore, the ratchet mechanism inhibits or prevents that the piston rod moves in the proximal direction.

The ratchet mechanism comprises a first and a second set of movable and stationary ratchet members. The movable ratchet body may be provided with movable ratchet members in the form of one or more tracks of sawtooth-shaped teeth and/or flexible arms. The same provisions apply for the stationary ratchet body. Therefore, the ratchet mechanism having at least two sets of teeth and arms is adapted for providing a first and a second correlated audible signal, in response to a relative movement between the movable and the stationary ratchet body, during the expelling of drug, which is typically a clicking sound. The signals are periodically generated during the expelling, however, in order to allow a user to distinguish between the audible sounds, they have to be separated in time, and therefore the first and the second correlated audible signals are adapted to be out of phase.

That the signals are correlated means that if one set of ratchet members provide a signal, during expelling, the second signal will also be generated at the same time or within a certain period from the first signal, if expelling is still ongoing.

That the signals are periodic means they are periodically generated with a characteristic frequency. The frequency of each signal may and may not be constant during delivery. The frequency may in particular decrease if a tension spring is used to drive the drive member, and if the tension spring cannot provide a constant torque during a complete delivery of a dose. It is however, preferred that the frequency is constant during delivery, and this can be obtained if the spring delivers a constant torque. That the signals are out of phase means that the clicking sounds are generated at different points in time, i.e., where the difference for any practical reasons can be measured with a microphone and ideally can be perceived by a user.

As the first and second signals are correlated with each other and with the speed of rotation of the ratchet body, the ratio between the frequency of each signal will be the same during delivery, if the rotation of the ratchet body is a pure rotation. If the ratio between the first and the second signal is 1 the frequency of each signal is the same, and if the ratio is 2, the frequency of the first signal is signal is twice the frequency of the second signal.

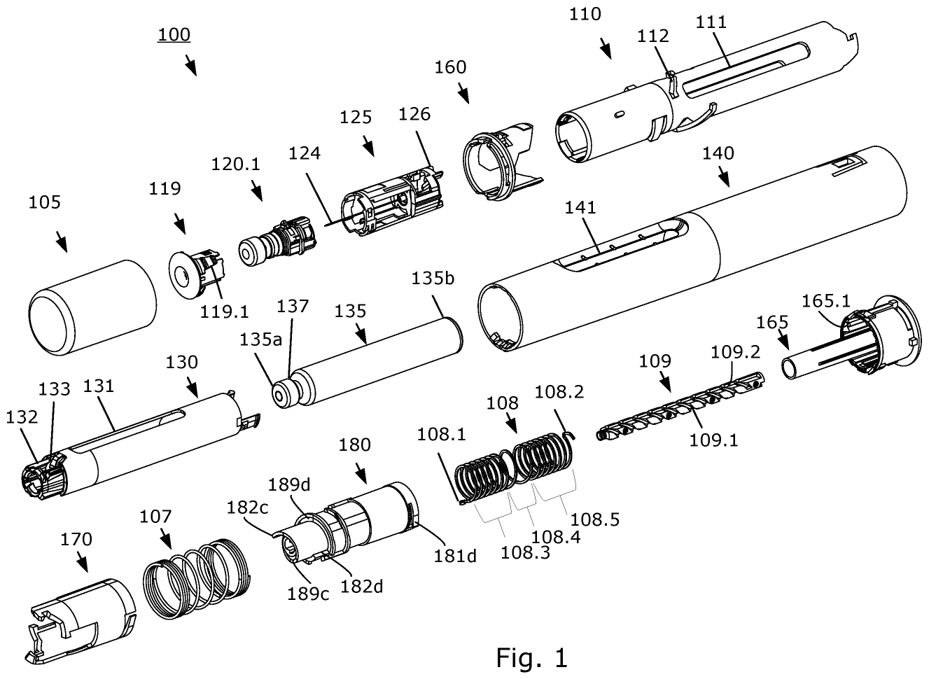

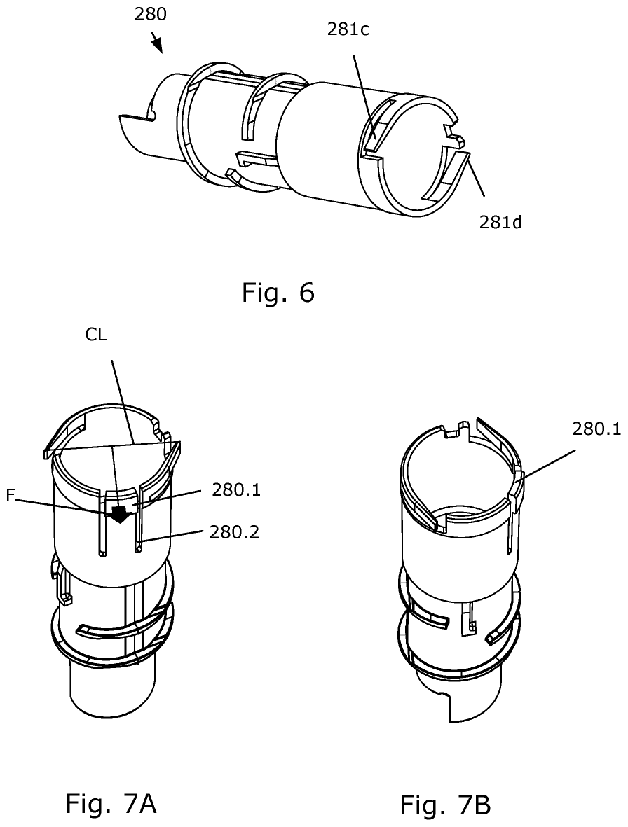

FIGS. 1-3 illustrate a first embodiment of a drug delivery device 100 for delivering a plurality of fixed doses of a medicament. The embodiment comprises a ratchet mechanism that may be modified according to the present disclosure. FIGS. 6-8 illustrate an embodiment of a safety assembly 300 with the ring member 390. The drug delivery devices 100 and 200 can both be modified to cooperate with the safety assembly 300.

European patent applications 19217357.3, 19217323.5, 19217333.4, 19217339.1, 19217358.1, 19217343.3 and 19217331.8 discloses further technical details of the device, which is not described in the present disclosure. The referenced patent applications are incorporated by reference.

Drug Delivery Device for Multiple Injections

FIG. 1 illustrates an exploded view of the drug delivery device 100. FIG. 1 illustrates a cap 105, a shield tip 119, a shield following portion 120.1 of a cleaning module, a needle hub 125 with a needle cannula 124, a housing insert portion 160, a tubular elongate needle shield structure 110, a cartridge holder 130, a cartridge 135, a tubular elongate housing structure 140, a connector 170, a shield return spring 107, a drive tube 180, a dose drive spring 108, a piston rod 109, and a spring base 165.

Housing Assembly

The drug delivery device comprises a housing assembly, providing a rigid frame with guides and connectors for guiding and connecting the other components of the device. The housing assembly comprises the housing insert portion 160, the tubular elongate housing structure 140, the cartridge holder 130 and the spring base 165. After final assembly these structures are fixedly connected. The elongate housing structure 140 comprises an internal thread for engaging an outer thread of the piston rod. The housing insert portion 160 comprises a cap snap at the end of a track for a bayonet coupling with the cap. The housing insert portion 160 further comprises a proximal edge for guiding the shield. The housing assembly may be referred to as the housing.

Needle Shield Assembly

The drug delivery device further comprises a needle shield assembly comprising the shield tip 119 and the elongate shield structure 110. The elongate shield structure 110 comprises a window 111 for inspection of the drug, the elongate shield can be arranged in a first position overlapping with the cartridge holder window 131, and in a second position with no overlap, wherein a solid portion of the elongate shield structure covers the window 131 in the second position. In this exemplified embodiment, the elongate shield structure 110 provides the activation member, which will be described in further details in the following. The needle shield assembly may be referred to as the needle shield. The elongate shield structure further comprises a step-wise helical guide structure 112, for turning rotational movement into axial movement, i.e., the step-wise helical guide is adapted to guide a helical movement of the shield in cooperation with structures or guides on the inner surface of the housing assembly.

Cartridge Holder

The cartridge holder 130 is adapted for receiving the cartridge 135. The cartridge holder comprises a window 131 for inspecting the drug in the cartridge 135. The cartridge holder 130 comprises a flexible arm for snapping to a neck portion 137 of the cartridge.

Cartridge

As further shown in FIG. 1A, the elongate cartridge 135 comprises a distal end 135a sealed by a pierceable septum and an open proximal end 135 closed by a piston. The piston is not shown on FIG. 1. The cartridge comprises a reservoir containing the plurality of fixed doses of a medicament. At the distal end 135a is provided a septum capped on by a cap. The cap and a main portion of the reservoir is separated by the neck portion 137.

Needle Assembly

The drug delivery device further comprises a needle assembly comprising a needle hub 125 and a reusable needle cannula 124. The cannula comprises a proximal end for piercing the pierceable septum and for establishing fluid communication with the reservoir, and a distal end for insertion into the skin of a subject or user of the device.

Piston Washer

A piston washer, although not shown on FIG. 1, can be connected to the piston rod to provide a pressure foot for contacting the piston. Alternatively, a dose measuring module for measuring the relative rotation between the piston rod and the piston can be provided between the piston rod and the piston. Such a measuring module also provides a suitable pressure foot. Such a dose measuring module is described in WO 20141128155, titled “Dose capturing cartridge module for drug delivery device. Alternatively, the piston rod directly contacts the piston”.

Cap

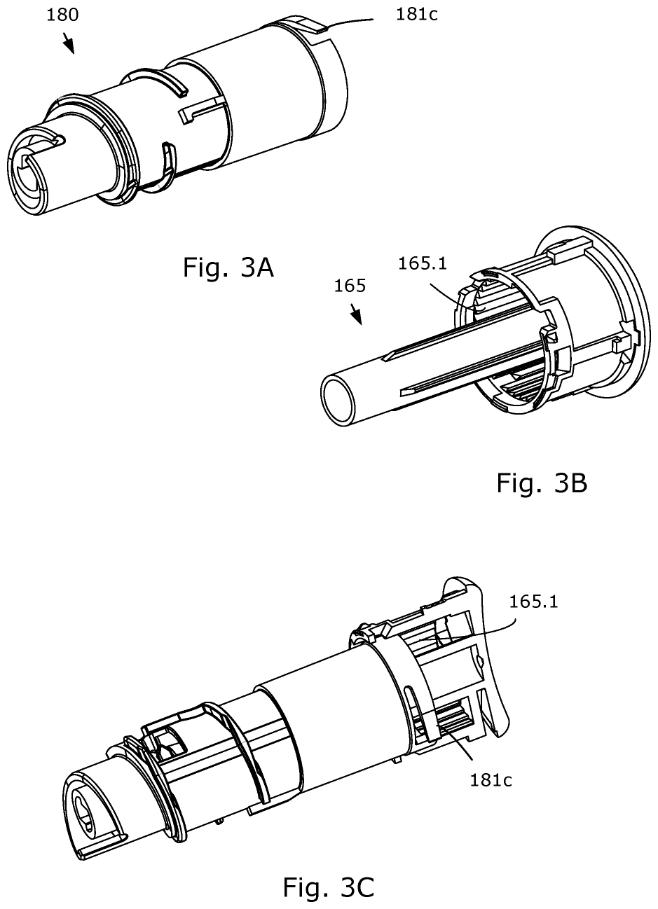

The cap 105 is adapted for releasable mounting to the housing insert portion 160. The cap comprises an inner surface with a protrusion adapted to be guided by the axial and the circumferential cap mount track 161 (FIG. 3A). The protrusion is further adapted to cooperate with a snap lock provided in the circumferential track 161, and thereby releasably lock the cap 105 to the inner housing portion 160. The cap is adapted to be mounted and demounted by a sequential axial and rotational movement, and thereby provides a bayonet coupling together with the drug delivery device. The inner surface of the cap 105 further comprises an axially extending rib (not shown) protruding from the inner surface and adapted for transferring a torque, during initialization, to the shield structure 110 through an axially extending rib 116 (FIG. 3A).

Spring Base

The spring base 165 is fixedly mounted to the housing structure 140 at the proximal end and is adapted to receive and support a compressible torsional drive spring 108. The spring base is tubularly shaped and comprises an inner surface with an angularly directed track of sawtooth-shaped teeth 165.1 providing a stationary ratchet body.

Drive Spring and Drive Tube

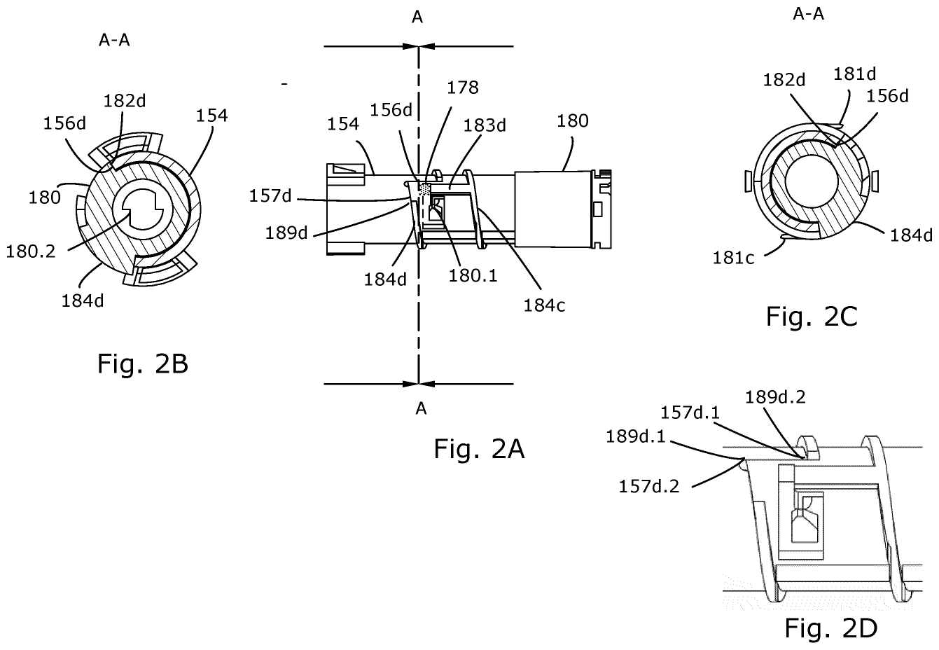

The drive spring 108 is pre-strained or winded up and positioned between the spring base 165 and the drive tube 180. The drive spring is further adapted to induce a torque on the drive tube 180. The drive tube 180 is axially splined to an axial track 109.2 of the piston rod 109 and provides a drive member adapted to expel the medicament upon rotation. The drive spring comprises torsional sections 108.3, 108.5, wherein the spacing between the coils is relatively small and a compressible section 108.4 adapted to transfer an axial force to the drive tube after compression and during expelling of the medicament. The ability to drive the drive tube in an axial direction together with an outer helical guide cooperating with the housing enables an end of dose mechanism, and enables a resetting of the drive tube. The drive tube comprises an axial portion 182, providing a rotational stop in a non-rotational position, and a helical portion 189 for guiding a helical movement in a rotational position. The drive tube further integrally comprises a movable ratchet body comprising a first 181c (tig. 2C) and a second 181d (FIG. 1 and FIG. 2C) flexible ratchet arm engaging the sawtooth-shaped teeth 165.1 of the spring base. To provide rotational stability the ratchet arms are symmetrically arranged, and are thereby adapted for providing in-phase periodic clicking sounds during delivery. The frequency of the clicking sounds may decrease during delivery due to relaxation of the spring.

Return Spring

The connector return spring 107 is positioned between the spring base 165 and the connector 170 and is adapted to urge the connector in the distal direction.

Cleaning Assembly

Cleaning the needle between injections allows the same integrated needle to be used a plurality of times in a clean condition. Therefore, in an alternative embodiment of the present disclosure, the drug delivery device comprises a cleaning assembly. The movable shield structure 110 is fixedly connected to the cleaning assembly through the shield following portion 120.1, and the principles of the cleaning module is disclosed in further details in WO2019/101670.

The shield can be arranged in different positions. An initial position defined by an initial angular position and a corresponding initial axial position. A locked position defined by a locked angular position and a corresponding locked axial position. An unlocked distal position defined by an unlocked angular position and a corresponding distal unlocked axial position. The movable shield can be changed by a combined rotational and proximal movement from the initial position to the locked position, wherein the shield is axially locked. In both positions the needle tip is covered by the shield and contained in the cleaning chamber assembly. During use the shield can be further rotated and moved further in the proximal direction in a helical movement to the unlocked distal position, whereby the tip is uncovered. By moving the shield further in the proximal direction in an axial movement the shield uncovers a larger portion of the needle and an injection can be made. After injection the shield is moved back to the locked position, whereby the needle tip is cleaned.

Activation Mechanism

FIG. 2A illustrates a perspective view of an inner tubular portion 154 of the housing 140 and the drive tube 180. The distal tubular portion 185 of the drive tube has been inserted into the inner tubular portion 154 of the housing, and is therefore not visible on the figure. FIG. 2B illustrates a cross section along the indicated line A-A and viewed from the proximal end. At the distal end, the drive tube 180 is provided with inward protrusions 180.2 protruding from an inner surface and adapted to engage the axial track 109.2 of the piston rod 109. FIG. 2C illustrates the cross section A-A viewed from the distal end. At the proximal end, a cross-section of the inner surface of the drive tube 180 is circular and the proximal end of the drive tube is adapted for accommodating the drive spring 108.

FIG. 2A further illustrates an activation tab 178 of the inner surface of the connector 170 (only the tab and not the rest of the connector is shown on FIG. 2A). The connector with the activation tab 178 is arranged in a position where it contacts a protruding tab 183 of the drive tube, and is thereby ready to transfer a proximal movement to the drive tube, whereby the drive tube can be activated. The drive tube 180 is biased by the drive spring 108 in a distal and counterclockwise direction. In FIG. 2A, the drive tube is illustrated in a rest or non-rotatable position, wherein the axial surface portion 182 abuts an axial surface portion 156 of the inner tubular portion 154 of the housing, and thereby prevents counterclockwise rotation of the drive tube 180. In the rest position the distal helical surface portion 182 of the drive tube also abuts the proximal helical surface portion 157 of the inner tubular portion 154 of the housing, and thereby prevents distal movement of the drive tube 180.

FIG. 2D illustrates in detail a proximal end of the helical surface portion 157d.1 defining a starting point of a helical dosing track and a distal end of the helical surface portion 157d.2 defining an ending point of the helical dosing track. Similarly, the distal helical surface portion 189d defines a front point or edge 189d.1 and a trailing point or edge 189d.2. In response to moving the connector in the proximal direction the activation tab 178, when arranged in in abutment with the protruding tab 183, induces a proximal movement of the drive tube 180. Thereby, the front edge 189d.1 is moved proximally along the axial surface portion 156 until it passes a proximal end of the axial surface portion and reaches the starting point 157d.1 of the helical dosing track. At this position the drive tube is positioned in a rotatable position, wherein the axial portions 182, 156 no longer abut. Due to the counterclockwise bias of the drive tube 189, the drive tube 189 can, in the rotatable position, start to rotate in the counterclockwise direction, and due the distal bias the front edge 189d.1 is forced into contact with the helical dose track of the inner tubular portion 154. The drive tube also comprises protruding helical structures 184c and 184d on the outer surface, which may cooperate with a housing structure to aid a distal movement during rotation. In the rotatable position, the drive tube with the front edge 189d.1 travels along the helical dose track 157 in a distal helical movement until it reaches the ending point 157d.2. The same effects are obtained by the angularly shifted axial surface portion 182c and distal helical surface portion 189c.

During rotation, the drive tube rotates the piston rod 109, which is threadably connected to the housing. Thereby, the piston rod 109 and the 136 are driven in the distal direction to expel an amount of medicament from the cartridge 135.

FIG. 3A, illustrates in perspective view the drive tube 180 with the flexible ratchet member 181c, and FIG. 3B illustrates the spring base 165 with the angularly directed track of saw-tooth shaped teeth 165.1. FIG. 3C, illustrates in perspective view the drive tube inserted into a tubular portion of the spring base. A portion of the spring base is broken away to illustrate the teeth 165.1. FIG. 3C illustrates the position of the drive tube relative to the spring base during the expelling of a drug, and also illustrates that upon rotation the ratchet arm 181c, will slide along the track of teeth. The symmetrically opposed ratchet arms 181c and 181d together with the saw-teeth 165.1 track provide a rachet mechanism.

During rotation the interaction between the movable ratchet body of the drive tube 180 and the stationary ratchet body of the spring base, i.e., the housing assembly, ensures unidirectional rotation of the piston rod, and provides the in-phase periodic clicking sounds or audible signals.

As appears, a single dose is delivered during a 360 degrees rotation of the drive tube 180. Therefore, the pitch of the threading between the piston rod 109 and the housing must be steep or high, if a relatively large amount of drug is required per dose.

Operation of the Injection Device for Multiple Injections of a Fixed Dose

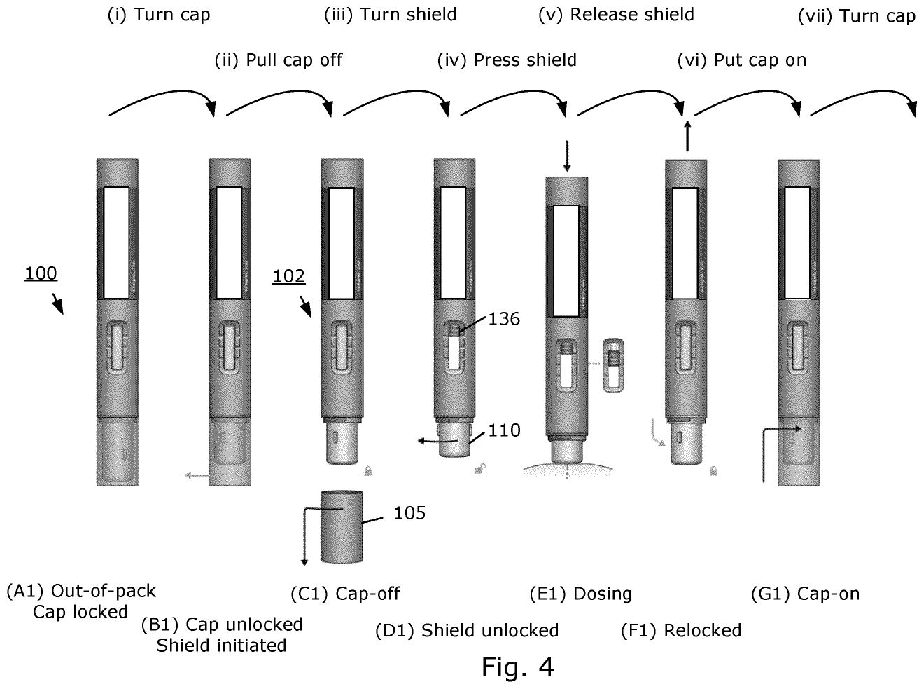

As illustrated in FIG. 4, when the user desires to take a first fixed dose, the drug delivery device is unpacked and thereby provided in the out-of-pack state (A1). Thereafter the drug delivery device is initiated by the user, by turning the cap in the counterclockwise direction. Hereby, the cap engages the needle shield, whereby the needle shield follows the rotation of the cap 105 until the cap 105 has been turned to a rotational stop. Due to the step-wise helical guide 112 of the shield, the needle shield is subject to a combined proximal and rotational movement, in response to the user turning the cap. Furthermore, by this initial rotation of the cap and the combined rotational and proximal movement of the needle shield, the needle cannula 124 pierces the septum of the cartridge 135, and thereby establishes fluid communication with a drug reservoir in the cartridge 135. Furthermore, in this operation the cartridge 135 is proximally displaced and pushed against the piston rod 109 or the piston washer 104. As the cannula has established fluid connection, and as the piston is arranged in abutment with the piston rod, the integrated needle is primed. As the cap reaches the rotational stop, the drug delivery device is positioned in the cap unlocked and initiated state (B1), wherein the cap is unlocked and positioned to be taken off.

In the following step the user pulls the cap 105 of, whereby the drug delivery device is arranged in the cap-off state (C1), and wherein the shield is locked against axial translation.

Hereafter, the user manually turns the shield in the counterclockwise direction, whereby the device is arranged in a shield unlocked state (D1), the shield is arranged in an unlocked position and can be pressed proximally into the housing. Due to the step-wise helical guide 112 of the shield, the shield is again subject to a combined proximal and rotational movement when operated between the cap-of state and the shield unlocked state. Hereby, the shield 110 connects with the connector 170.

Hereafter, the user presses the needle shield against the injection site, whereby the shield and the connector 170 is proximally displaced against the force of the shield return spring 107. Hereby, the needle is inserted into the skin or subcutaneous layer of a patient. By this operation, the axial movement of the shield triggers the drive mechanism, and a fixed dose is delivered through the needle cannula in a dosing state (E1). At the end of dose, the piston 136 (FIG. 4) has moved to the next position, which is indicated by the fixed dose residual scale on the housing, and the drug delivery device can be removed from the injection site. The cut-out window of the residual scale shows the piston in the next position.

After the dose has been completed, the user removes the device from the skin, and the pressure is thereby released from the shield. Consequently, the shield moves in the distal direction due to the action of the return spring 107. Due to step-wise helical guide 112 of the shield, the shield is subject to a distal movement followed by a combined distal and rotational movement, whereby the shield automatically returns to a relocked state (F1).

Hereafter, the user puts on the cap 105 by an axial movement to put the device in a capon state (G1), which is the last state shown in the sequence shown in FIG. 2. The cap unlocked state and the cap on state, within the same sequence, differs technically in that the cartridge comprises a dose less in the cap on state. Finally, the cap is turned, and thereby snap locked to the housing assembly.

A subsequent dose can be administrated in a similar manner, but without requiring initialization. When the last dose has been administered, it is not possible to activate the drive mechanism again.

The multiuse fixed dose device described above holds four doses with same fixed dose volume. Each dose can be ejected by one full turn of the integrated drive spring. The drive spring is not rewound between ejections. Thus, the available torque is lower for each dose. Consequently, each of the four ejections take longer time than the previous.

In order to provide acoustic feedback to the user the engine is equipped with a click arm that rotates with the rotating engine parts. The free end of the arm is spring loaded against stationary saw tooth shaped ratchet teeth arranged in a circular pattern on a stationary part.

With the number and size of teeth that are typically fitted into the available space combined with the relatively low speed of the later and particularly the last of the four doses the clicking sound has been found to be too slow.

To some extent the clicking speed can be increased by fitting a larger number of smaller/shorter teeth. Because there are practical limits to how small teeth can be made the achievable increase of clicking speed is limited in the traditional and typical setting described above. Therefore, the present disclosure concerns identifying solutions wherein the clicking speed can be increased beyond the limit explained above.

The basic idea of a solution according to the present disclosure is to use two click arms arranged with an angular distance between the two tips corresponding to half the angular pitch of the stationary rachet teeth plus an angle equivalent to a number of complete teeth (the number may be 0). In this way the number of clicks generated during one rotation of the drive tube, will be two times the number of teeth on the angular track.

The number of clicks could be further increased by using more than two arms arranged in a similar manner. I.e. the number of clicks could be tripled by using three arms.

Drive Member with Ratchet Members Out-of-Phase

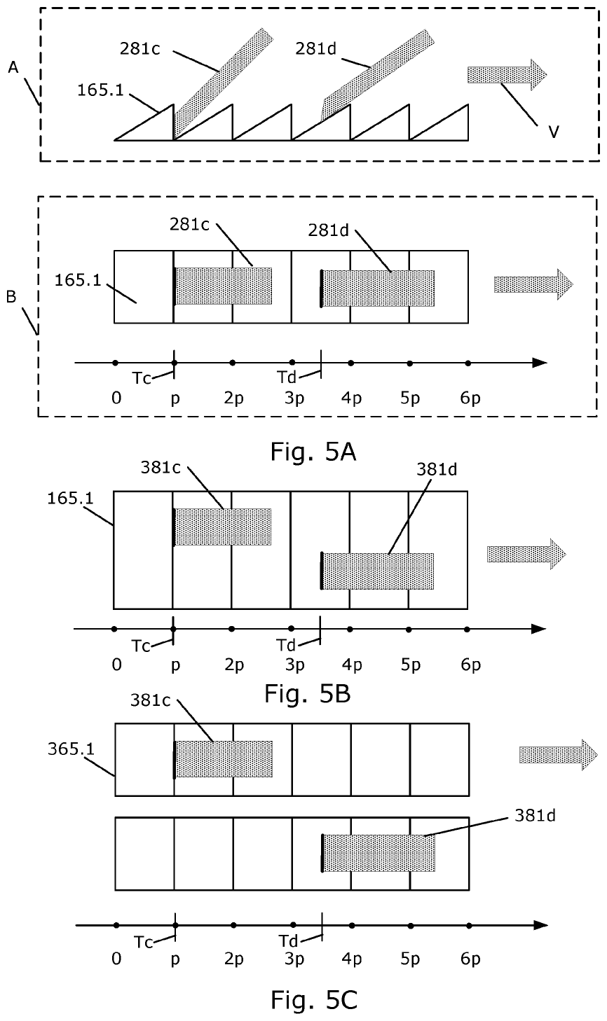

FIG. 5A illustrates the working principle of a first embodiment of a drive tube 280 with movable ratchet members 281c and 281d, wherein the angular distance between the tips of the two ratchet members corresponds to half the angular pitch of the stationary rachet teeth 165.1 plus an angle equivalent to a number of complete teeth 165.1. FIG. 6 illustrates a perspective view of the drive tube 280. In this illustrated embodiment one of the ratchet arms has been shifted a small angle, e.g., a half tooth, from rotational symmetry. In this case the number of teeth of equal size could be even, e.g., 24.

Alternatively, the pitch of the teeth are changed adapted and adapted to allow the movable ratchet members to remain in two-fold rotational symmetry, as seen in FIG. 3. In this case the number of teeth could be uneven, e.g., 25.

FIG. 5A Box A, illustrates schematically a track of angularly directed saw-tooth shaped teeth. The teeth are represented as “un-filled” triangles with an inclined moderately increasing straight curve from the right and followed by an abrupt steeply decreasing straight curve. The track is “unfolded” and represented as an axially directed track, and viewed as a cross-section. The movable ratchet members 281 are patterned, and a an arrow V with the same pattern, indicates that the ratchet members 281 are moving relative to the non-filled saw-tooth shaped track. The pitch is here defined as the axial length of each tooth, and is indicated with a p. For an angularly directed track the pitch would correspondingly be represented as the arch length of a tooth on the inner surface of the spring base. In FIG. 5A, Tc indicates the position, where the tip of the ratchet member 281c contacts the teeth, and Td indicates the position, where the tip of the ratchet member 281d contacts the teeth. As noted, there is 2.5 times the pitch or 2.5 teeth between the tip of the ratchet arm 281c positioned at p, and the tip of ratchet arm 281d positioned at 3.5p. This is an arbitrary number, which is chosen for illustrative purposes only. However, an embodiment with only 2.5 teeth between the tips would be possible, but they would not be positioned with two-fold rotational symmetry, as this would result in a very large pitch with very few clicks per revolution. As the movable ratchet body with the movable ratchet members 281 starts to rotate, the ratchet members will periodically move up and down, and they will periodically provide audible signals, e.g., clicking or snapping sounds, each time the ratchet member snaps from the top to the bottom of the saw-tooth track. However, as there is not an integer number of teeth between the contact points of the ratchet members, the ratchet members will be out-of-phase in their periodic behaviour. In the illustrated example, the distance between the tips is an integer number of teeth plus a half pitch, which means that the generated audible signals are in anti-phase. However, the distance could also be an integer number plus 0.1, 0.2 . . . 0.9 times the pitch. In all cases, the signal generation of the two ratchet members 281 is out of phase. However, for practical reasons and for being able to distinguish between the first and the second signal, it is required that the relation between the “degree” of out-of-phase-position of the ratchet members 281 and the speed of rotation is balanced, i.e., the additional fraction of a pitch, and the speed of rotation of the drive tube 280, is balanced to make it possible to distinguish the clicking sounds. A relatively small degree of being out-of-phase may be measured with a microphone, but for practical reasons the clicking sound should be perceivable by the human ear. The audible clicking sounds are correlated, as the ratchet members 281 on the movable ratchet body 280 are provided on the same body. Therefore, the audible sounds they generate will also be correlated and the frequency of the signals will depend on the rotational speed of the rotationally movable ratchet body.

Alternatively, a single ratchet member in the form of a ring of teeth is provided on an outer surface of the movable rachet body, and 2 ratchet members are provided on the stationary body. The audible signals generated by such a system will also be correlated.

Positioning the ratchet arms 281 at the same axial or longitudinal position, limits the axial or longitudinal length of the track of angularly oriented saw-teeth 265.1.

The inventors of the present invention discovered, that due to radial play between the drive tube 280 and the inner surface of the spring base 165, the drive tube 280 is able to move radially in an unpredictable pattern causing the timing between the two sets of clicks to vary and their common impression to appear less desirable. That is to say the clicking frequency is periodic and dependent on the pitch of the teeth and the speed of rotation, but due to the radial play and the almost diametrically positioned ratchet members 281 a variation of the frequency is introduced, which may be less desirable. This effect can be observed as a variation in each of the first and second signals corresponding to each of the first 281c, 265.1 and second set 281d, 265.1 of ratchet members. If either of these signals varies to have time intervals of increasing and decreasing frequency it can be an indication of influence by radial play. In particular if the frequency of one of the signals increases this is an indication of influence from radial play.

The inventors of the present invention discovered that when a radial play is provided between the movable ratchet body and the housing, the frequency of each of the signals may vary in an increasing and decreasing manner. Therefore, to prevent this variation the movable ratchet body (280, 380, 480) or the housing is adapted to provide a biasing radial reaction force between the movable ratchet body and the housing to counteract influence by the radial play, and thereby to minimize a variation of a frequency of the first periodic signal or the second periodic signal.

FIGS. 7A and 7B illustrate a further development of the embodiment of FIG. 6, wherein influence of radial play has been limited or eliminated by adding an integrated spring 280.1 acting with a force (F) radially and perpendicularly to a connection line (CL) between the tips of the two arms. As best seen on FIG. 7B, a small angularly extending section 280.1 of the drive tube 280 extends radially to limit influence of the play, and two slits 280.2 extending axially from the proximal end of the drive tube 281 increases radial flexibility of the section or spring element 280.1, i.e., the slits 280.2 provide the spring force (F) perpendicular to the connection line (CL). The section 280.1 extends further in the radial direction than the remaining section of the drive tube, i.e., the section 280.1 has a larger outer radius than the remaining section of the drive tube 280.

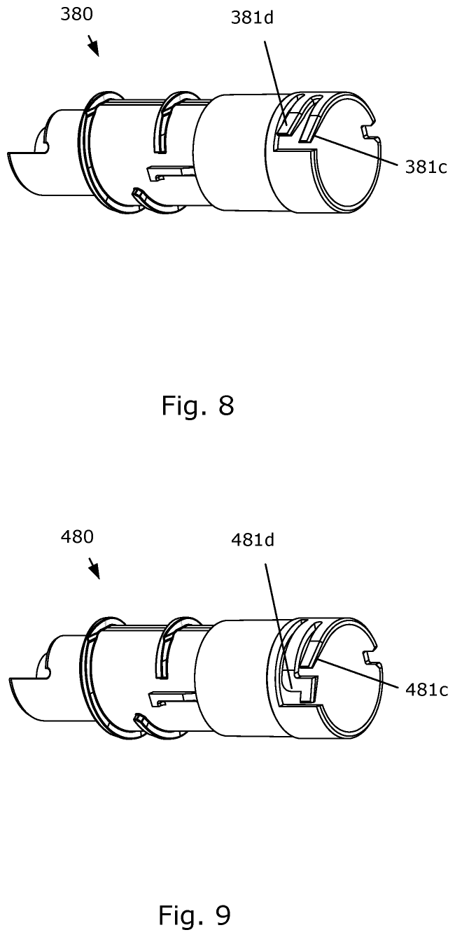

Similarly to FIG. 5A, FIG. 5B illustrates the working principle of an alternative embodiment of a drive tube 380. The drive member 380, is illustrated in perspective view in FIG. 8. The drive tube 380 comprises two ratchet members within a small angular fraction, i.e., there is a small angular offset between the tips of the two ratchet members. This arrangement practically eliminates the undesired influence on the impression of the combined click sounds from radial movement of the drive tube, which was seen for the embodiment of FIG. 5A, wherein the click arms were almost positioned with a 180 degrees distance and without a spring element. In the illustrated example, the distance between the tips is 2.5, which means that the generated audible signals are in anti-phase. However, the distance could also be an integer number plus 0.1, 0.2 . . . 0.9 times the pitch. The integer number in the illustrated example of FIG. 8, is preferably 0, 1, 2, 3, 4, 5, 6, 7, 8, or 9, and the fraction of a pitch is preferably 0.5. The drive tube may be 1 cm in diameter and the pitch may be defined by 24 equally sized teeth spanning the circumference of the inner surface. Such an embodiment, eliminates for practical reasons the influence of radial play, as the ratchet members 281 functions as a spring element urging the drive tube in the radial direction and pushes it towards the side surface of the housing.

Although radial play influence is almost eliminated, the embodiment may additionally be provided with a spring element, as shown in FIGS. 6A and 6B, if a small amount undesired impression of clicking sounds remain.

FIG. 5C illustrates the working principle of an alternative embodiment of FIGS. 5B and 8, wherein a single track of saw-tooth shaped teeth 165.1 is divided into two tracks 365.1, or a number of saw-tooth tracks corresponding to the number of axially separated ratchet members 381.

As another or additional alternative to providing an angular off-set between the ratchet members 381, an angular off-set could be provided between the teeth.

As another or additional alternative the pitch of the teeth could be different between the two tracks, whereby the frequency between the generated signals would be different. However, the ratio between the frequency of the first and second signal would be constant.

Permanent deflection of one of the two arms during storage might cause relaxation of the arm. This can be solved by removing the one tooth the arm in question is resting against during storage. As the first and second clicks are practically simultaneous and different from the following clicks removal of the tooth has no practical influence on the sound.

Therefore, as another or additional alternative, one of the teeth can be removed to allow both ratchet members 281 to be in a rest position during storage.

FIG. 9 illustrates an alternative embodiment, wherein the drive tube is provided with two flexible members 481 with in a small angle or angular section, i.e., within a few number teeth. The working principle is illustrated in FIG. 5A. The flexible member 481c is formed by a straight flexible arm, whereas the second flexible member 481d is formed by a bended flexible arm, whereby the tips of the two flexible members are axially aligned. Hereby, is obtained a drive tube, wherein the requirement to the axial extension of the saw-tooth track is limited and the influence of radial play is reduced.

The solutions are also relevant for other motorized injection devices operating with high viscosity liquids and or long and thin injection needles.

The invention according to the present disclosure could also be implemented in an alternative fixed dose device as described in WO04078239 and additionally provided with a ratchet mechanism between the drive member (screw ring) and the housing. For such an alternative drug delivery device, the dose delivery mechanism comprises a piston rod axially splined to the housing and a drive member threadably engaged with an outer thread of the piston rod. When the drive member is rotated, the rotationally fixed but axially movable piston rod is pushed forward. Additionally the drive member should be adapted to provide a movable ratchet body, and the housing should be provided with a stationary ratchet body to ensure one-way rotation. Additionally, the movable or the stationary ratchet body should be provided with flexible arms and the other body with one or more tracks of saw-tooth shaped teeth. Additionally, the flexible arms should be off-set to provide out of phase clicking sounds.

The invention could also be implemented in a drug delivery device with an adjustable dose as described in WO 14161952. In an embodiment according to the present disclosure such a drug delivery device is additionally provided with off-set ratchet arms to provide out of phase clicking sounds.

The invention could also be implemented in a drug delivery device as described in WO 99/38554 in relation to FIG. 6-10, wherein the piston rod is threaded to the housing and an axially movable push button. As the push button is pushed, in a pure axial movement, the piston rotates and advances. A piston rod guide axially splined to the piston rod is rotated by the piston during the expelling of a drug. The piston rod guide is provided with a ratchet to ensure unidirectional movement. Therefore, in an embodiment according to the present disclosure the piston rod guide rotated and driven by the rotating and advancing piston rod is additionally provided with off-set ratchet arms to provide out of phase clicking sounds.

### Further Exemplary Embodiments

In an exemplary embodiment is provided a drug delivery device 100 for delivering an amount of medicament, wherein the device comprises:
a housing 140, 165, 365;
a medicament reservoir 135 with a piston 136;
a drive mechanism comprising: (i) a piston rod 109 for driving the piston 136 during a distal movement and thereby expelling the amount of medicament, (ii) a drive member 280, 380, 480 operationally arranged for driving the piston rod 109, and a rotatably arranged movable ratchet body 280, 380, 480, wherein the movable ratchet body 280, 380, 480 is operatively connected to the piston rod 136 and adapted to rotate relative to the housing 140, 165, 365.

The housing comprises a stationary ratchet body 165.1, 365.1 arranged to cooperate with the movable ratchet body 280, 380, 480 and thereby provide a ratchet mechanism allowing a ratcheted guiding of the piston rod 136 during the expelling of drug.

The ratchet mechanism comprises a first and a second set of movable and stationary ratchet members 281, 381, 481, 165.1, 365.1 adapted to provide a first and a second plurality of periodic audible signals, in response to a relative movement between the movable and the stationary ratchet body, during the expelling of medicament.

The ratchet mechanism is adapted to generate the first and second plurality of periodic signals out-of-phase, whereby each of the signals are distinguishable by a user.

In a further aspect of the embodiment, the piston rod 109 is in threaded engagement with the housing and the drive member 280, 380, 480 is axially splined to the piston rod 109, wherein a rotational movement of the drive member 280, 380, 480 induces an axial movement of the piston rod 109 during the expelling of medicament.

Alternatively, the piston rod 109 is axially splined to the housing and the drive member 280, 380, 480 is in threaded engagement with the piston rod 109 during the expelling of medicament.

In further aspect of the embodiment, the movable ratchet body is provided as a portion of the drive member 280, 380, 480, wherein the audible signals are generated by rotation of the drive member 280, 380, 480 together with the piston rod.

In further aspect of the embodiment, the movable ratchet body 280 comprises a spring element 280.1 providing a radial force against the housing, and thereby counteract the influence of radial play between the movable ratchet body 280 and the housing. Alternatively the housing comprises a spring element acting on the drive member.

In further aspect of the embodiment, tips of the ratchet members 281 of the movable ratchet body 280 are positioned within an angular distance between 170 and 190 degrees.

Alternatively, tips of the ratchet members 381, 481 of the movable ratchet body 380, 480 are positioned within an angular distance of 45 degrees to provide a radial force against the housing, and thereby counteract the influence of radial play between the movable ratchet body 380, 480 and the housing.

In further aspect of the embodiment, tips of the movable ratchet members 281, 481 are axially aligned, whereby tip contact points on the stationary ratchet member 165.1 are axially aligned.

Alternatively, the piston rod 109 is in threaded engagement with the housing and the drive member is in threaded engagement with the piston rod 109, wherein an axial movement of the drive member induces a rotational and axial movement of the piston rod 109 during the expelling of medicament.

In further aspect of the embodiment, the movable ratchet body is provided as a portion of a piston rod guide axially splined to the piston rod 109, wherein the audible signals are generated by rotation of the piston rod 109 together with the piston rod guide.

In further aspect of the embodiment, the movable ratchet body comprises a spring element providing a radial force against the housing, and thereby eliminates the influence of radial play between the movable ratchet body and the housing.

In further aspect of the embodiment, the ratchet mechanism is adapted to inhibit proximal movement of the piston rod.

In further aspect of the embodiment, the drive mechanism is adapted to generate the periodic signals with a constant ratio between the frequencies, to provide a consistent sound generation during expelling.

In further aspect of the embodiment, the drive mechanism is adapted to generate the periodic signals with the same frequency, to provide a consistent sound generation during expelling.

In further aspect of the embodiment, the drive mechanism is adapted to generate the periodic signals in anti-phase, to provide a consistent sound generation during expelling and a homogeneous resolution in time.

In further aspect of the embodiment, the drive mechanism additionally comprises a third set of movable and stationary ratchet members 281, 381, 481, 165.1, 365.1 adapted to provide a third plurality of periodic audible signals, in response to a relative movement between the movable and the stationary ratchet body, during the expelling of medicament. The ratchet mechanism is adapted to generate the first, second and third plurality of periodic signals out-of-phase, whereby each of the signals are distinguishable by a user.

In further aspect of the embodiment, the drive mechanism is adapted to generate the periodic signals with a homogeneous resolution in time, to provide a consistent sound generation during expelling.

In further aspect of the embodiment, the movable ratchet members 281, 381, 481 comprises a flexible arm with a tip for engaging one or more stationary ratchet member 165.1, 365.1, wherein each of the one or more stationary ratchet members comprises a ring of teeth.

In further aspect of the embodiment, the stationary ratchet members comprise a flexible arm with a tip for engaging one or more movable ratchet members, wherein each of the one or more movable ratchet members comprises a ring of teeth.

In a further aspect the drive mechanism is adapted for delivering a fixed dose during a rotation between 340 and 360 degrees, and wherein the piston rod is adapted to advance between 1 and 2 cm, whereby the pitch of the piston rod is relatively high.

In another exemplary embodiment is provided a method of using a drug delivery device 100 for delivering an amount of medicament, wherein the device comprises:
a housing 140, 165, 365;
a medicament reservoir 135 with a piston 136;
a drive mechanism comprising: a piston rod 109 for driving the piston 136 during a distal movement and thereby expelling the amount of medicament, a drive member 280, 380, 480 operationally arranged for driving the piston rod 109, and a rotatably arranged movable ratchet body 280, 380, 480, wherein the movable ratchet body 280, 380, 480 is operatively connected to the piston rod 136 and adapted to rotate relative to the housing 140, 165, 365.

The housing comprises a stationary ratchet body 165.1, 365.1 arranged to cooperate with the movable ratchet body 165.1, 365.1 and thereby provide a ratchet mechanism allowing a ratcheted guiding of the piston rod 136 during the expelling of drug.

The ratchet mechanism comprises a first and a second set of movable and stationary ratchet members 281, 381, 481, 165.1, 365.1 adapted to provide a first and a second plurality of periodic audible signals, in response to a relative movement between the movable and the stationary ratchet body, during the expelling of medicament.

The method comprises activating or driving the drive mechanism and thereby generate the first and second plurality of periodic signals out-of-phase, whereby each of the signals are distinguishable by a user.

Although the exemplary embodiments only illustrate drug delivery devices with injection needles, the invention can also be implemented in drug delivery devices connectable with an infusion set instead of a needle. The invention according to the present disclosure is suitable for both durable and prefilled/single-use devices.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery device for delivering an amount of medicament, wherein the device comprises:
a housing;
a medicament reservoir with a piston;
a drive mechanism comprising: a piston rod for driving the piston during a distal movement and thereby expelling the amount of medicament, a drive member operationally arranged for driving the piston rod, and a rotatably arranged movable ratchet body, wherein the movable ratchet body is operatively connected to the piston rod and adapted to rotate relative to the housing during delivery of the medicament,
wherein the housing comprises a stationary ratchet body arranged to cooperate with the movable ratchet body and thereby provide a ratchet mechanism allowing a ratcheted guiding of the piston rod during the expelling of drug;
wherein the ratchet mechanism comprises a first and a second set of movable and stationary ratchet members adapted to provide a first and a second plurality of periodic audible signals, corresponding to the first and the second set of ratchet members, in response to a relative rotational movement between the movable and the stationary ratchet body, during the expelling of medicament;
wherein the ratchet mechanism is adapted to generate the first and second plurality of periodic signals out-of-phase, whereby each of the signals are distinguishable by a user, wherein a radial play is provided between the movable ratchet body and the housing, and
wherein the movable ratchet body or the housing is adapted to provide a biasing radial reaction force between the movable ratchet body and the housing to counteract influence by the radial play, and thereby to reduce a variation of a frequency of the first periodic signal or the second periodic signal.

2. A drug delivery device according to claim 1, wherein tips of the ratchet members of the movable ratchet body are positioned within an angular distance of 45 degrees to provide a radial force against the housing, and thereby counteract the influence of the radial play.

3. A drug delivery device according to claim 1, wherein tips of the ratchet members of the movable ratchet body are positioned within an angular distance of 10 degrees to provide a radial force against the housing, and thereby counteract the influence of the radial play.

4. A drug delivery device according to claim 1, wherein the movable ratchet body comprises a spring element providing a radial force against the housing, and thereby counteracts the influence of the radial play.

5. A drug delivery device according to claim 1, wherein the housing comprises a spring element providing a radial force against the movable ratchet body, and thereby counteracts the influence of the radial play.

6. A drug delivery device according to claim 1, wherein the piston rod is in threaded engagement with the housing and the drive member is axially splined to the piston rod, wherein a rotational movement of the drive member induces an axial movement of the piston rod during the expelling of medicament.

7. A drug delivery device according to claim 1, wherein the piston rod is axially splined to the housing and the drive member is in threaded engagement with the piston rod, wherein a rotational movement of the drive member induces an axial movement of the piston rod during the expelling of medicament.

8. A drug delivery device according to claim 1, wherein the movable ratchet body is the drive member, wherein the audible signals are generated by rotation of the drive member together with the piston rod.

9. A drug delivery device according to claim 1, wherein tips of the ratchet members of the movable ratchet body are positioned within an angular distance between 170 and 190 degrees.

10. A drug delivery device according to claim 1, wherein tips of the movable ratchet members are axially aligned, whereby tip contact points on the stationary ratchet member are axially aligned.

11. A drug delivery device according to claim 1, wherein the piston rod is in threaded engagement with the housing and the drive member is in threaded engagement with the piston rod, wherein an axial movement of the drive member induces a rotational and axial movement of the piston rod during the expelling of medicament, wherein the movable ratchet body is provided as a portion of a piston rod guide axially splined to the piston rod, wherein the audible signals are generated by rotation of the piston rod together with the piston rod guide.

12. A drug delivery device according to claim 1, wherein the ratchet mechanism is adapted to allow one-directional rotation of the movable ratchet body, and thereby inhibit proximal movement of the piston rod.

13. A drug delivery device according to claim 1, wherein the drive mechanism is adapted to generate the periodic signals with a constant ratio between the frequency of the first periodic signal and the second periodic signal.

14. A drug delivery device according to claim 1, wherein the drive mechanism is adapted to generate the first and the second periodic signals with the same frequency.

15. A drug delivery device according to claim 1, wherein the drive mechanism is adapted to generate the periodic signals in anti-phase, to provide a consistent sound generation during expelling and a homogeneous resolution in time.
